(19) United States
(12) Patent Application Publication
(10) Pub. No.: US 2005/0287614 A1
(43) Pub. Date: Dec. 29, 2005

(54) Assay for detecting an analyte containing or labelled with a haem moiety

(76) Inventor: William Hunter Symonds, Salisbury (GB)

Correspondence Address: JOHN S. PRATT, ESQ;KILPATRICK STOCKTON, LLP, 1100 PEACHTREE STREET, ATLANTA, GA, 30309, US

(21) Appl. No.: 10/519,738

(22) PCT Filed: Jun. 25, 2003
(86) PCT No.: PCT/GB03/02716

§ 371 Date: Dec. 28, 2004

(30) Foreign Application Priority Data
Jun. 28, 2002 (GB) 0214947.4

(51) Int. Cl. G01N 33/53

(52) U.S. Cl. 435/7.92

(57) ABSTRACT

A method for detecting an analyte containing or labelled with a haem moiety within a sample, said method comprising: a) contacting said sample with a magnetic bead having immobilised thereon a specific binding partner for said analyte and allowing analyte to bind to said specific binding partner; b) separating the magnetic beads from the sample, and if necessary, labelling the immobilised analyte with a haem containing label; c) resuspending the beads and subjecting them to alkaline conditions sufficient to release haem moieties therefrom but not to extract inorganic iron from the beads; d) detecting released haem moieties using a luminol chemiluminescent assay procedure.

The present invention relates to an assay method, in particular an assay for detecting an analyte containing a haem moiety within a sample.

The haem group is a prosthetic group associated with certain cellular proteins. It is built around an atom of iron, and it is conveniently detected using a light emitting luminol chemiluminescent reaction.

In this reaction, luminol (3-aminophthalazide), or a functional chemiluminescent derivative thereof, is oxidised by an oxidant in a basic aqueous solution to generate a light emitting species (3-aminophathalate) as illustrated.

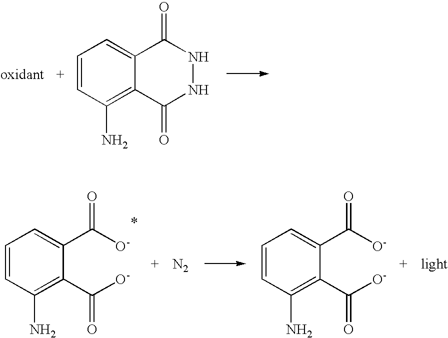

The reaction is catalysed by metal cations, in particular, the “organic” iron, present in haem molecules, to increase light emission or to increase the speed of oxidation of luminol to the light emitting species and therefore the onset or intensity of light production.

It may be used therefore in assays for the haem molecule. Functional chemiluminescent derivatives of luminol are known in the art.

However, the reaction may be highly sensitive to a variety of contaminants, and therefore separation of analyte from the source of possible contaminants is highly desirable. In particular, inorganic iron can also initiate this light emitting reaction. It has been reported for example, that Fe(III) interferes positively with the reaction at some concentrations and negatively at others, whilst Fe(II) shows significant positive interference (Yuan J et al., Anal. Chem. 1999, 71, 1975-1980).

Consequently, it is generally recognised that the inorganic iron contaminants are particularly undesirable in a solution that is being tested in this way and could lead to false positive or negative results.

Magnetic bead separation is a particularly useful way of concentrating analytes within a sample. Specific binding partners for a particular analyte may be immobilised on the ferromagnetic beads, which are then contacted with a sample suspected of containing the analyte. Analyte becomes bound to the beads, which may then be separated from the bulk sample using magnetic separation methods, to attract the ferromagnetic beads. Once separated, analyte can be released from the beads in a more concentrated form, and detected.

However the conditions required to release a haem moiety from the analyte-bead complex is generally chemically quite stringent, and it is expected that these would be accompanied by extraction of at least some inorganic iron from the beads.

Such separation methods therefore are contraindicated for use in the luminol reaction, since the contamination risk from inorganic iron is significantly higher.

The applicants have found however, that this combination can be successfully carried out.

According to the present invention there is provided a method for detecting an analyte containing or labelled with a haem moiety within a sample, said method comprising:

a) contacting said sample with a magnetic bead having immobilised thereon a specific binding partner for said analyte and allowing analyte to bind to said specific binding partner;

b) separating the magnetic beads from the sample, and if necessary, labelling the immobilised analyte with a haem containing label;

c) resuspending the beads and subjecting them to alkaline conditions sufficient to release haem moieties therefrom but not to extract inorganic iron from the beads;

d) detecting released haem moieties using a luminol chemiluminescent assay procedure.

In particular, step (c) is conducted within a pH range of from 12.5-13.5. This is suitably achieved using a buffer or a working solution which is preferably the working solution of alkaline luminol. This solution suitably combines NaOH that causes the release of the haem moiety and luminol or an equivalent functional chemiluminescent reagent in a single solution.

The applicants have found that step (d) may be carried out directly on the bead suspension. The nature of the light emitted is so strong, that the presence of the beads does not detract from the signalling process. However, if desired, after step (c), the magnetic beads can be separated, and step (d) is carried out on supernatant remaining.

Suitably the beads are subjected to one or more washing steps between step (b) and step (c). In these, the magnetic beads are resuspended in a washing solution, and thereafter, separated from the washing solution.

Resuspension of beads during these washing steps, as well as during step (c) may be carried out using conventional methods, such as by using a whirlimixer, but is preferably carried out using relatively gentle methods such as pipetting, in order to minimise loss of bound material from the beads.

The method of the invention is particularly suitable for the detection of analytes such as spores, in particular Bacillus globigii(BG) spores, which may be more difficult to detect using other assay methods, because of the relative difficulty of accessing cellular materials, in particular proteins such as enzymes.

Alternatively, where the target analyte does not contain a haem moiety, it may be labelled with a haem containing second specific binding partner such as an antibody. Some enzymatic labels commonly used in immunoassays, such as horseradish peroxidase (HRP) contain a haem prosthetic group. Using the method of the invention, a non-haem containing analyte may be a protein or peptide, which is bound to an antibody-coated bead. After initial capture, an HRP labelled antibody is added to the separated beads after step (b) to introduce a haem label onto any immobilised analyte. It is then necessary to separate and resuspend the beads, optionally with a washing step between steps (b) and (c). In this embodiment, the haem moiety is extracted from the antibody which forms a “sandwich” with any analyte immobilised on the beads.

Suitably, in step (d), luminol is first added to the released haem moieties in the alkaline conditions of the buffer, suitably in excess, and incubated with them, and thereafter, oxidant added in a sufficient quantity to generate a signal. Suitable oxidants include those known in the art, including peroxides, such as hydrogen peroxide, perborate, permanganate or hypochlorite salts, for example of alkali metals such as sodium or potassium, or iodine. Preferably the oxidant used is a perborate, and in particular, sodium perborate. Another preferred oxidant is hydrogen peroxide.

The oxidant and the luminol, or functional chemiluminescent derivative thereof, are suitably added in a significant amount compared to the likely concentration of haem in the solution.

This means that the signal generated can be related to the amount of haem present in the sample acting as a catalyst for the reaction, and the magnitude of the signal is not limited by the lack of luminol or oxidant.

For instance, where for example a 0.005% w/w luminol solution is used in the reaction, the ratio of the volume of the luminol solution added to the reaction: the volume of reagent solution is in the range of from 1:2 to 10:1 and preferably about 1:1. Where the luminol solution used is of a different concentration, the volume ratio will be varied accordingly to provide equivalent relative amounts of the reactants.

The amount of oxidant added will depend upon the particular oxidant used, but it should suitably be sufficient to oxidise all of the luminol present in the reaction.

Immobilisation of specific binding partners such as antibodies or binding fragments thereof, onto the magnetic beads may be carried out using any of the conventional procedures. Step (a) is suitably carried out by incubating the coated beads with a solution of the sample for a sufficient period of time, and at a suitable temperature, for example at about 37° C., to allow good capture of the analyte by the specific binding partner.

Suitably, the concentration of magnetic beads used is sufficient to ensure good capture efficiency of the analyte. Thus the concentration of beads used is suitably in the range of from about 1×10⁴ bead.ml¹ to 1×10⁸ beads.ml⁻¹

Similarly, the amount of liquid added during the resuspension in step (c) is kept low in order to provide improved concentration factors.

The method of the invention has been found to have very good sensitivity, in particular for spores as analytes. Furthermore, the strength of the signal obtainable in this way means that no amplification step is required, so it provides a very rapid assay. In addition, it may be detected by a wide range of detectors, including photodiodes.

Reagent costs for this assay are very low compared to say bioluminescent assay systems.

Furthermore, by coupling the assay to the IMS capture, the inherent susceptibility of the chemiluminescent assay to interferents is reduced.

The invention further provides a kit for carrying out the method of the invention. In particular the kit will comprise magnetic beads, luminol, or functional chemiluminescent derivatives thereof and a working solution having a pH within the range of from 12.5-13.5.

The working solution is preferably the working solution of alkaline luminol and may be a buffer. This solution suitably combines the NaOH required for the release of the haem moiety and the luminol or functional chemiluminescent reagent in a single solution.

Suitably, the magnetic beads are coated with a specific binding partner for an analyte. Particular examples of specific binding partners include antibodies or binding fragments thereof. In addition, the kit may further comprise an oxidant for luminol as described above, and in particular, sodium perborate.

As described hereinafter, Dynal tosylated beads were coated with CBD rabbit anti-BG antibody and used to capture Bacillus globigii spores. A chemiluminescent assay end point was successfully used as an alternative to those that produce a bioluminescent signal.

Surprisingly, initial tests on “naked” beads indicated that un-coated beads did not give significant blank readings with the chemiluminescent assay. Later tests with antibody coated beads did produce higher readings than the blank measurements (re-suspension buffer). It was found that the background signals were not prohibitively high, even when doing the assay in a PMT based luminometer, which is generally regarded as being over-sensitive for chemiluminescence work.

The invention will now be particularly described by way of example.

### EXAMPLE

### Method and Materials

Instrumentation

All luminometric measurements were made in a TL Plus Luminometer from Thermo Life Sciences (Basingstoke, UK), using 3.5 mL polystyrene tubes obtained from Biotrace (Bridgend, UK). 1-second delay interval and 1 second measurement duration was used in all measurements.

Reagents

Luminol was obtained from Fluka (Poole, UK), EDTA and sodium perborate was obtained from BDH (Poole, UK), sodium hydroxide, phosphate buffer, tris buffer, PBS Tween buffer were obtained from Sigma (Poole, UK). Sterile distilled water and sterile phosphate buffered saline were obtained from Gibco (Paisley, UK). Tryptone soya agar plates were obtained from Oxoid (Basingstoke, UK). 280 μm tosyl and epoxy activated paramagnetic beads were obtained from Dynal (UK) Ltd (Wirrall, UK). Rabbit anti-BG polyclonal antibodies and rabbit anti-E coli polyclonal antibodies were obtained from DSTL Detection & Diagnostics antibody group.

A stock solution of alkaline luminol (100 g sodium hydroxide, 37.5 g EDTA, 5 g luminol dissolved in 1 litre of sterile distilled water) was prepared and kept in a (light proof) container at 4° C. Working solution of alkaline luminol was prepared by diluting 8 ml of the above to 100 ml with water. Sodium perborate solution was prepared by dissolving 1 g of sodium perborate and 0.1 g EDTA in 100 ml of sterile distilled water.

Paramagnetic beads were coated using the manufacturers' coating procedures. Antibody conjugation to the tosyl-activated beads was performed in Buffer A (100 mM sodium phosphate buffer, pH 7.4). Conjugation was performed for 24 hours at 37° C. using a Dynal mixing wheel.

Immunomagnetic Separation

A 10-fold dilution series in PBS was prepared from stock BG spore suspension at a concentration of 1.0E+11 cfu.ml⁻¹. 1000 μl of the test dilution was dispensed into sterile Eppendorf tubes. Anti-BG coated magnetic beads were added to each sample to give a final concentration of ˜1.0E+07 beads.ml⁻¹. Anti-E coli coated magnetic beads were added to duplicate samples as a control. The samples were incubated at 37° C. for 10 minutes on a Dynal mixing wheel (18 rpm).

On removal from the mixing wheel the samples were placed into a Dynal Magnetic Particle Concentrator (MPC) and the magnet was applied. After 2 minutes the unbound supernatant was removed (and retained for plate count assays) after which the magnet was removed from the rack.

The beads were then resuspended by the gentle addition of 100 μl of PBS or PBS containing 0.05% Tween 20. The magnet was applied to the rack and after 2 minutes the unbound supernatant was removed and discarded.

The magnet was then removed from the rack and the beads were resuspended in 1000 μl PBS, although the final resuspension volume was varied depending on the final assay requirements.

Assay

100 μl aliquots of the bead suspensions resulting from the immunomagnetic separation protocol were removed and placed into 3.5 mL polystyrene tubes. 100 μl of the working luminol solution were added to the sample and were incubated (at room temperature) for 1 minute. 100 μl of sodium perborate solution were added and the light generated by the reaction was immediately measured in the luminometer.

Plate Counts

Bacterial CFU counts on the samples, were performed by plating out 100 μL of the sample on to tryptone soya agar plates in triplicate and incubating these for 24 hours at 37° C. before counting.

Using the combination of an immuno-magnetic capture and separation with a chemiluminescent endpoint assay, a specific detection limit of 6.0E+06 cfu/ml unwashed BG spore was demonstrated.

1. A method for detecting an analyte containing a haem moiety within a sample, the method comprising:
a) contacting the sample with one or more magnetic beads having immobilised thereon a specific binding partner for the analyte and allowing the analyte to bind to the specific binding partner;
b) separating the magnetic beads from the sample ,
c) subjecting the beads to alkaline conditions sufficient to release haem moieties from the beads; and
d) detecting released haem moieties using a luminol chemiluminescent assay procedure.

2. The method of claim 1 wherein step (c) is conducted within a pH range of from 12.5 to 13.5.

3. The method of claim 1 wherein the beads are resuspended in step (c) and step (d) is carried out directly on the bead suspension.

4. The method of claim 1 wherein the beads are resuspended in step (c); after step (c) the magnetic beads are separated from the suspension; and step (d) is carried out on the separated suspension.

5. The method of claim 1 wherein between step (b) and step (c), the magnetic beads are resuspended in a washing solution, and thereafter, separated from the washing solution.

6. The method claim 1 wherein the analyte is a spore.

7. The method of claim 6 wherein the analyte is a Bacillus spore.

8. The method of claim 1 wherein in step (c) the alkaline conditions are sufficient to release haem moieties without extracting inorganic iron from the beads.

9. (canceled)

10. The method of claim 1 wherein in step (d) luminol is added to and incubated with the released haem moieties and oxidant added in a sufficient amount to generate the a signal.

11. The method of claim 10, wherein the amount of oxidant present is sufficient to oxidise all of the luminol.

12. The method of claim 10 wherein the oxidant is sodium perborate or hydrogen peroxide.

13. The method of claim 1 wherein the specific binding partner for the analyte is an antibody or binding fragment thereof.

14. A kit for detecting an analyte in a sample, wherein the analyte contains a haem moiety or is labelled with a haem moiety, comprising magnetic beads, luminol or functional chemiluminescent derivatives thereof and a working solution having a pH within the range of from 12.5 to 13.5.

15. The kit of claim 14 wherein the magnetic beads are coated with a specific binding partner for an analyte.

16. The kit of claim 15 wherein the specific binding partner is an antibody.

17. The kit of claim 14 which further comprises an oxidant.

18. The kit of claim 17 wherein the oxidant is sodium perborate or hydrogen peroxide.

19. A method for detecting an analyte labelled with a haem moiety within a sample. the method comprising:
a) contacting the sample with one or more magnetic beads having immobilised thereon a specific binding partner for the analyte and allowing the analyte to bind to the specific binding partner;
b) separating the magnetic beads from the sample;
c) subjecting the beads to alkaline conditions sufficient to release the haem moiety from the beads: and
d) detecting the released haem moiety using a luminol chemiluminescent assay procedure.

20. The method of claim 19 wherein the haem moiety is a horseradish peroxidase labelled antibody specific for the analyte.
